(19) United States
(12) Patent Application Publication
(10) Pub. No.: US 2018/0125422 A1
(43) Pub. Date: May 10, 2018

(54) APPARATUS AND METHOD FOR PROVIDING HEALTH STATUS OF CARDIOVASCULAR SYSTEM

(71) Applicant: SAMSUNG ELECTRONICS CO., LTD., Suwon-si (KR)

(72) Inventors: Dae Geun JANG, Yongin-si (KR); Seung Woo NOH, Seongnam-si (KR)

(73) Assignee: SAMSUNG ELECTRONICS CO., LTD., Suwon-si (KR)

(21) Appl. No.: 15/475,808

(22) Filed: Mar. 31, 2017

(30) Foreign Application Priority Data
Nov. 7, 2016 (KR) 10-2016-0147600

(51) Int. Cl. A61B 5/00; A61B 5/024; A61B 5/029; A61B 5/021

(52) U.S. Cl. CPC A61B 5/681; A61B 5/6831; A61B 5/02108; A61B 5/029; A61B 5/02438

(57) ABSTRACT

An apparatus and a method for providing a health status of a cardiovascular system are provided. The apparatus includes a body oscillation measurer configured to measure body oscillation data, and a processor configured to estimate a health status of a cardiovascular system, based on the measured body oscillation data.

### CROSS-REFERENCE TO RELATED APPLICATION

This application claims priority from Korean Patent Application No. 10-2016-0147600, filed on Nov. 7, 2016 in the Korean Intellectual Property Office, the disclosure of which is incorporated herein by reference in its entirety.

### BACKGROUND

### 1. Field

Apparatuses and methods consistent with example embodiments relate to an apparatus and method for providing a health status of a cardiovascular system.

### 2. Description of Related Art

Healthcare technology has attracted much attention due to the rapid entry into an aging society and relevant social problems such as increase in medical expenses. Accordingly, not only medical devices that can be utilized by hospitals and inspection agencies but also small-sized medical devices that can be carried by individuals such as wearable devices are being developed.

In addition, such a small-sized medical device is worn by a user in the form of a wearable device capable of directly measuring cardiovascular health status such as blood pressure or the like, so that the user can directly measure and manage cardiovascular health status.

Therefore, research is being conducted for a new technique that allows a person without professional knowledge to continuously measure and be provided with cardiovascular health status in a non-intrusive/unobstructive manner, without any additional equipment manipulation.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to an aspect of an example embodiment, there is provided an apparatus for providing a health status of a cardiovascular system, the apparatus including a body oscillation measurer configured to measure body oscillation data, and a processor configured to estimate a health status of a cardiovascular system, based on the measured body oscillation data.

The body oscillation measurer may be further configured to measure the body oscillation data, using any one or any combination of an acceleration sensor, a piezoelectric film, a load cell, a radar, and a photoplethysmogram sensor.

The health status of the cardiovascular system may include any one or any combination of a blood pressure, a cardiac output, a blood vessel elasticity, and a peripheral resistance.

The processor may include a segmentator configured to divide the measured body oscillation data into predetermined time intervals, a feature calculator configured to calculate a feature from the divided body oscillation data, and a health status estimator configured to estimate the health status of the cardiovascular system, using the calculated feature and a cardiovascular system health status estimation model.

The feature may include any one or any combination of a signal energy, a sum of absolute values of signals, a sum of envelope values, a number of peaks, a standard deviation of amplitudes, and a number of level crossings.

The apparatus may further include a contact pressure measurer configured to measure contact pressure data of the apparatus in contact with a body part.

The contact pressure measurer may be further configured to measure the contact pressure data, using any one or any combination of an acceleration sensor, a piezoelectric film, a load cell, a radar, and a photoplethysmogram sensor.

The processor may include a body oscillation calibrator configured to calibrate the measured body oscillation data, based on the measured contact pressure data, a segmentator configured to divide the calibrated body oscillation data into predetermined time intervals, a feature calculator configured to calculate a feature from the divided body oscillation data, and a health status estimator configured to estimate the health status of the cardiovascular system, using the calculated feature and a cardiovascular system health status estimation model.

The body oscillation calibrator may be further configured to search a calibration coefficient database to determine a body oscillation calibration coefficient corresponding to the measured contact pressure data, and adjust any one or any combination of a gain, a slope, and an offset of the measured body oscillation data, based on the determined body oscillation calibration coefficient.

The processor may include a segmentator configured to divide the measured body oscillation data into predetermined time intervals, a feature calculator configured to calculate a feature from the divided body oscillation data, a feature calibrator configured to calibrate the calculated feature, based on the measured contact pressure data, and a health status estimator configured to estimate the health status of the cardiovascular system, using the calibrated feature and a cardiovascular system health status estimation model.

The feature calibrator may be further configured to search a calibration coefficient database to determine a feature calibration coefficient corresponding to the measured contact pressure data, and adjust any one or any combination of a gain, a slope, and an offset of the calculated feature, based on the determined feature calibration coefficient.

The apparatus may further include an actuator configured to adjust a contact pressure of the apparatus in contact with the body part, and the processor may include a contact pressure controller configured to generate a control signal for driving the actuator such that the measured contact pressure data reaches a predetermined contact pressure. The body oscillation measurer may be further configured to measure the body oscillation data in response to the measured contact pressure data reaching the predetermined contact pressure. The processor may further include a segmentator configured to divide the measured body oscillation data into predetermined time intervals, a feature calculator configured to calculate a feature from the divided body oscillation data, and a health status estimator configured to estimate the health status of the cardiovascular system, using the calculated feature and a cardiovascular system health status estimation model.

The apparatus may be implemented as a wearable device.

According to an aspect of another example embodiment, there is provided a method of providing a health status of a cardiovascular system, the method including measuring body oscillation data, and estimating a health status of a cardiovascular system, based on the measured body oscillation data.

The health status of the cardiovascular system may include any one or any combination of a blood pressure, a cardiac output, a blood vessel elasticity, and a peripheral resistance.

The method may further include dividing the measured body oscillation data into predetermined time intervals, and calculating a feature from the divided body oscillation data. The estimating may include estimating the health status of the cardiovascular system, using the calculated feature and a cardiovascular system health status estimation model.

The feature may include any one or any combination of a signal energy, a sum of absolute values of signals, a sum of envelope values, a number of peaks, a standard deviation of amplitudes, and a number of level crossings.

The method may further include measuring contact pressure data of an apparatus in contact with a body part, calibrating the measured body oscillation data, based on the measured contact pressure data, dividing the calibrated body oscillation data into predetermined time intervals, and calculating a feature from the divided body oscillation data. The estimating may include estimating the health status of the cardiovascular system, using the calculated feature and a cardiovascular system health status estimation model.

The method may further include measuring contact pressure data of an apparatus in contact with a body part, dividing the measured body oscillation data into predetermined time intervals, calculating a feature from the divided body oscillation data, and calibrating the calculated feature, based on the measured contact pressure data. The estimating may include estimating the health status of the cardiovascular system, using the calibrated feature and a cardiovascular system health status estimation model.

The method may further include measuring contact pressure data of an apparatus in contact with a body part, adjusting a contact pressure of the apparatus in contact with the body part such that the measured contact pressure data reaches a predetermined contact pressure. The measuring may include measuring the body oscillation data in response to the measured contact pressure data reaching the predetermined contact pressure. The method may further include dividing the measured body oscillation data into predetermined time intervals, and calculating a feature from the divided body oscillation data. The estimating may include estimating the health status of the cardiovascular system, using the calculated feature and a cardiovascular system health status estimation model.

### BRIEF DESCRIPTION OF THE DRAWINGS

The above and/or other aspects will be more apparent by describing example embodiments with reference to the accompanying drawings, in which:

FIG. 1 is a block diagram illustrating an apparatus for providing a health status of the cardiovascular system, according to an example embodiment;

FIG. 2 is a block diagram illustrating an apparatus for providing a health status of a cardiovascular system, according to another example embodiment;

FIG. 3 is a block diagram illustrating an apparatus for providing a health status of a cardiovascular system, according to still another example embodiment;

FIG. 4 is a block diagram illustrating an apparatus for providing a health status of a cardiovascular system, according to yet another example embodiment;

FIG. 5 is a block diagram illustrating an apparatus for providing a health status of a cardiovascular system, according to still another example embodiment;

FIG. 6 is a block diagram illustrating an apparatus for providing a health status of a cardiovascular system, according to yet another example embodiment;

FIG. 7 is a flowchart illustrating a method of providing a health status of a cardiovascular system, according to an example embodiment;

FIG. 8 is a flowchart illustrating a method of providing a health status of a cardiovascular system, according to another example embodiment;

FIG. 9 is a flowchart illustrating a method of providing a health status of a cardiovascular system, according to still another example embodiment;

FIG. 10 is a flowchart illustrating a method of providing a health status of a cardiovascular system, according to yet another example embodiment;

FIG. 11 is a flowchart illustrating a method of providing a health status of a cardiovascular system, according to still another example embodiment;

FIG. 12 is a flowchart illustrating a method of providing a health status of a cardiovascular system, according to yet another example embodiment; and

FIG. 13 is a perspective view of a wrist wearable device.

### DETAILED DESCRIPTION

The following detailed description is provided to assist the reader in gaining a comprehensive understanding of the methods, apparatuses and/or systems described herein. Various changes, modifications, and equivalents of the systems, apparatuses and/or methods described herein will suggest themselves to those of ordinary skill in the art. In the following description, a detailed description of known functions and configurations incorporated herein will be omitted when it may obscure the subject matter with unnecessary detail.

In some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

Terms described in below are selected by considering functions in example embodiments and meanings may vary depending on, for example, a user or operator's intentions or customs. Therefore, in the following example embodiments, when terms are specifically defined, the meanings of terms may be interpreted based on definitions, and otherwise, may be interpreted based on meanings recognized by those skilled in the art.

As used herein, the singular forms are intended to include the plural forms as well, unless the context clearly indicates otherwise. It will be further understood that the terms “comprises” and/or “comprising,” or “includes” and/or “including” when used in this description, specify the presence of stated features, numbers, steps, operations, elements, components or combinations thereof, but do not preclude the presence or addition of one or more other features, numbers, steps, operations, elements, components or combinations thereof.

It will also be understood that the elements or components in the following description are discriminated in accordance with their respective main functions. In other words, two or more elements may be made into one element or one element may be divided into two or more elements in accordance with a subdivided function. Additionally, each of the elements in the following description may perform a part or whole of the function of another element as well as its main function, and some of the main functions of each of the elements may be performed exclusively by other elements. Each element may be realized in the form of a hardware component, a software component, and/or a combination thereof.

Throughout the drawings and the detailed description, unless otherwise described, the same drawing reference numerals will be understood to refer to the same elements, features, and structures. The relative size and depiction of these elements may be exaggerated for clarity, illustration, and convenience.

An apparatus for providing a health status of the cardiovascular system described herein may be implemented as a software module or in the form of a hardware chip and be mounted in an electronic device. In this case, the electronic device may include a mobile phone, a smart phone, a notebook computer, a personal digital assistant (PDA), a portable multimedia player (PMP), a navigation system, an MP3 player, a digital camera, a wearable device, etc., and the wearable device may include various types of wearable devices, such as a wristwatch type, a wristband type, a ring type, a belt-type, a necklace type, an ankle band type, a thigh band type, a forearm band type, and the like. However, the electronic device is not limited to the above mentioned examples, and the wearable device is also not limited to the above-described examples.

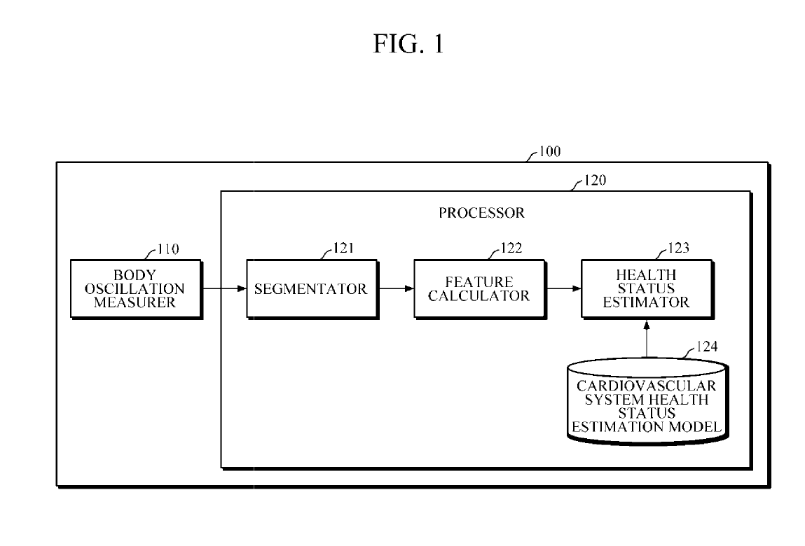

FIG. 1 is a block diagram illustrating an apparatus 100 for providing a health status of a cardiovascular system, according to an example embodiment.

Referring to FIG. 1, the apparatus 100 for providing a health status of a cardiovascular system includes a body oscillation measurer 110 and a processor 120.

The body oscillation measurer 110 may measure user's body oscillation data. Here, the body oscillation may refer to an oscillation caused by the blood flow or pulsation of the user's body. According to an example embodiment, the body oscillation measurer 110 may measure the body oscillation data using an acceleration sensor, a piezoelectric film, a load cell, a radar, a photoplethysmogram (PPG) sensor, or the like.

The processor 120 may estimate a health status of the user's cardiovascular system on the basis of the measured body oscillation data. Here, the health status of the cardiovascular system may refer to information related to the cardiovascular system, such as a blood pressure, a cardiac output, a blood vessel elasticity, and a peripheral resistance.

In addition, the processor 120 may perform preprocessing to remove noise from the body oscillation data using various noise cancellation algorithms.

The processor 120 may include a segmentator 121, a feature calculator 122, a health status estimator 123, and a cardiovascular system health status estimation model 124.

The segmentator 121 may divide the measured body oscillation data into predetermined time intervals. Here, the predetermined time may be set to a time interval (e.g., 5 seconds) or may be set to a user's input value or a default value in consideration of the heartbeat cycle.

The feature calculator 122 may calculate a feature from the divided body oscillation data. Here, the feature may refer to an oscillation characteristic of the body oscillation and may include a signal energy, a sum of absolute values of signals, a sum of envelope values, a number of peaks, a standard deviation of amplitudes, a number of level crossings, and the like. When a level in level crossing becomes 0, the level crossing may be zero crossing.

The health status estimator 123 may estimate the health status of the user's cardiovascular system using the calculated feature and the cardiovascular system health status estimation model 124.

The cardiovascular system health status estimation model 124 may define a relationship between at least one feature and each health status of the cardiovascular system. The cardiovascular system health status estimation model 124 may be implemented in the form of a mathematical algorithm or a matching table, but is not limited to any particular form.

The cardiovascular system health status estimation model 124 may be built in advance and stored in a storage device. In this case, the storage device may include any one or any combination of a flash memory type, a hard disk type, a multimedia card micro type, a card type memory (e.g., SD or XD memory), a random access memory (RAM), a static random access memory (SRAM), a read only memory (ROM), an electrically erasable programmable read only memory (EEPROM), a programmable read only memory (PROM), a magnetic memory, a magnetic disk, and an optical disk.

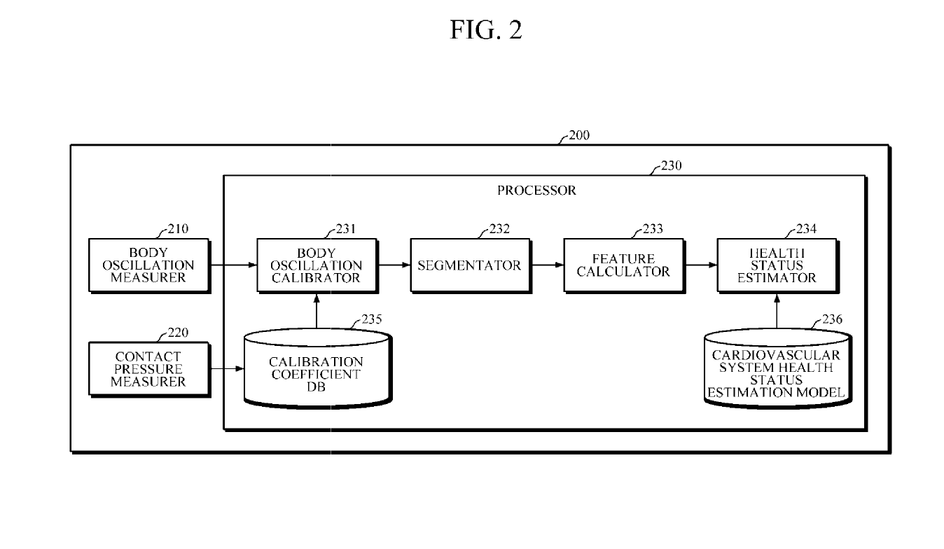

FIG. 2 is a block diagram illustrating an apparatus 200 for providing a health status of a cardiovascular system, according to another example embodiment.

Referring to FIG. 2, the apparatus 200 for providing a health status of a cardiovascular system includes a body oscillation measurer 210, a contact pressure measurer 220, and a processor 230.

The body oscillation measurer 210 may measure a user's body oscillation data. According to an example embodiment, the body oscillation measurer 210 may measure the body oscillation data using the acceleration sensor, a piezoelectric film, a load cell, radar, a PPG sensor, or the like.

The contact pressure measurer 220 may measure contact pressure data of the apparatus 200 in contact with the user's body part. According to an example embodiment, the contact pressure may measure the contact pressure data using an acceleration sensor, a piezoelectric film, a load cell, radar, a PPG sensor, or the like.

The processor 230 may calibrate the measured body oscillation data on the basis of the measured contact pressure data and estimate the health status of the user's cardiovascular system on the basis of the calibrated body oscillation data. In addition, the processor 230 may perform preprocessing to remove noise from the body oscillation data using various noise cancellation algorithms.

The processor 230 may include a body oscillation calibrator 231, a segmentator 232, a feature calculator 233, a health status estimator 234, a calibration coefficient database (DB) 235, and a cardiovascular system health status estimation model 236.

The body oscillation calibrator 231 may calibrate the measured body oscillation data measured by the body oscillation measurer 210 on the basis of the contact pressure data measured by the contact pressure measurer 220. For example, the body oscillation calibrator 231 may search the calibration coefficient DB 235 to calculate a body oscillation calibration coefficient that corresponds to contact pressure data, and may adjust a gain, a slope, and an offset of the body oscillation data on the basis of the calculated body oscillation calibration coefficient.

The segmentator 232 may divide the calibrated body oscillation data into predetermined time intervals. Here, the predetermined time may be set to a time interval (e.g., five seconds) or may be set to a user's input value or a default value in consideration of the heartbeat cycle.

The feature calculator 233 may calculate a feature from the divided body oscillation data, and the health status estimator 234 may estimate the health status of the user's cardiovascular system using the calculated feature and the cardiovascular system health status estimation model 236.

The calibration coefficient DB 235 may be a set of data defining relationships between contact pressures and body oscillation calibration coefficients and may be experimentally derived and established in advance.

The cardiovascular system health status estimation model 236 may define relationships between at least one feature and each cardiovascular system health status. The cardiovascular system health status estimation model 236 may be implemented in the form of a mathematical algorithm or a matching table, but is not limited to any particular form.

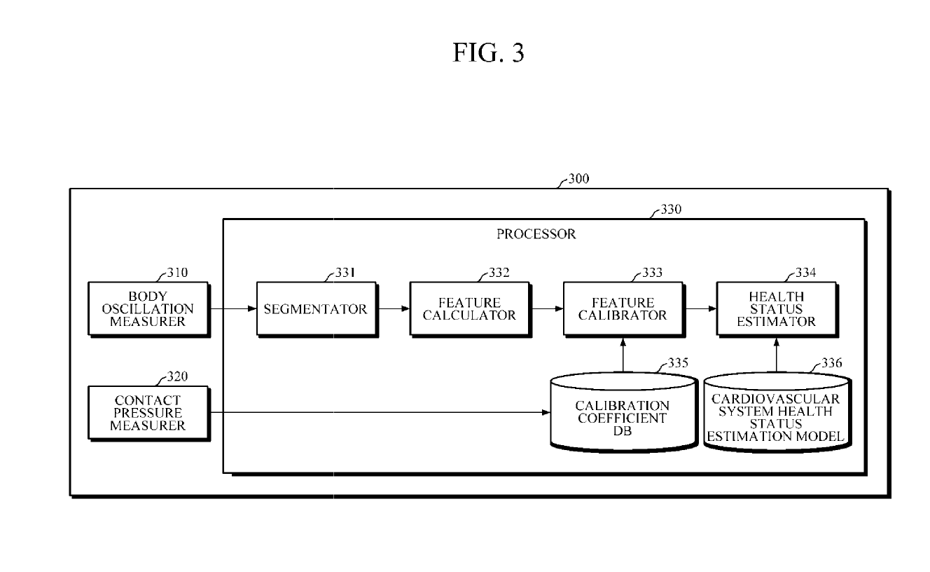

FIG. 3 is a block diagram illustrating an apparatus 300 for providing a health status of a cardiovascular system, according to still another example embodiment.

Referring to FIG. 3, the apparatus 300 for providing a health status of a cardiovascular system includes a body oscillation measurer 310, a contact pressure measurer 320, and a processor 330.

The body oscillation measurer 310 may measure a user's body oscillation data, and the contact pressure measurer 320 may measure contact pressure data of the apparatus 300 in contact with the user's body part. For example, the body oscillation measurer 310 and the contact pressure measurer 320 may measure the body oscillation data and the contact pressure data using an acceleration sensor, a piezoelectric film, a load cell, radar, a PPG sensor, or the like, respectively.

The processor 330 may calibrate a feature calculated from the measured body oscillation data on the basis of the measured contact pressure data and estimate the health status of the user's cardiovascular system on the basis of the calibrated feature. In addition, the processor 330 may perform preprocessing to remove noise from the body oscillation data using various noise cancellation algorithms.

The processor 330 may include a segmentator 331, a feature calculator 332, a feature calibrator 333, a health status estimator 334, a calibration coefficient DB 335, and a cardiovascular system health status estimation model 336.

The segmentator 331 may divide the measured body oscillation data into predetermined time intervals. Here, the predetermined time may be set to a time interval (e.g., five seconds) or may be set to a user's input value or a default value in consideration of the heartbeat cycle.

The feature calculator 332 may estimate a feature from the divided body oscillation data.

The feature calibrator 333 may calibrate the feature calculated by the feature calculator 332 on the basis of the contact pressure data measured by the contact pressure measurer 320. For example, the feature calibrator 333 may search the calibration coefficient DB 335 to calculate a feature calibration coefficient that corresponds to the measured contact pressure data and may adjust a gain, a slope, an offset, and the like of the feature on the basis of the calculated feature calibration coefficient.

The health status estimator 334 may estimate the health status of the user's cardiovascular system using the calibrated feature and the cardiovascular system health status estimation model 336.

The calibration coefficient DB 335 may be a set of data defining relationships between contact pressures and body oscillation calibration coefficients and may be experimentally derived and established in advance.

The cardiovascular system health status estimation model 336 may define relationships between at least one feature and each cardiovascular system health status. The cardiovascular system health status estimation model 336 may be implemented in the form of a mathematical algorithm or a matching table, but is not limited to any particular form.

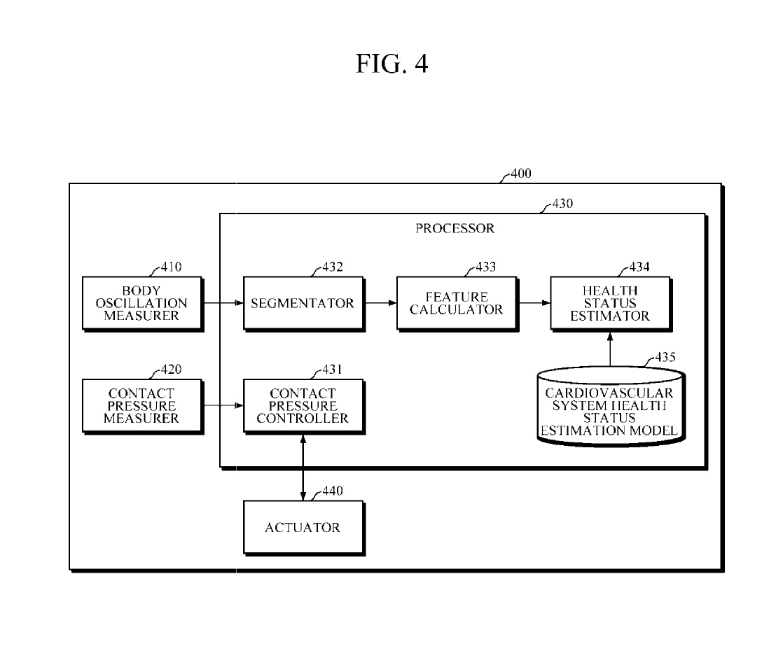

FIG. 4 is a block diagram illustrating an apparatus 400 for providing a health status of a cardiovascular system, according to yet another example embodiment.

Referring to FIG. 4, the apparatus 400 for providing a health status of a cardiovascular system includes a body oscillation measurer 410, a contact pressure measurer 420, a processor 430, and an actuator 440.

The body oscillation measurer 410 may measure a user's body oscillation data, and the contact pressure measurer 420 may measure contact pressure data of the apparatus 400 in contact with the user's body part.

The processor 430 may generate a control signal for driving the actuator 440 such that the contact pressure of the apparatus 400 for the user's body part reaches a predetermined contact pressure. In addition, the processor 430 may estimate the health status of the user's cardiovascular system on the basis of the body oscillation data measured at the time when the contact pressure of the apparatus 400 reaches the predetermined contact pressure. Also, the processor 430 may perform preprocessing to remove noise from the body oscillation data using various noise cancellation algorithms.

The processor 430 may include a contact pressure controller 431, a segmentator 432, a feature calculator 433, a health status estimator 434, and a cardiovascular system health status estimation model 435.

The contact pressure controller 431 may generate a control signal for driving the actuator 440 such that the contact pressure of the apparatus 400 for the user's body part reaches a predetermined contact pressure. In this case, the predetermined contact pressure may be experimentally derived as an optimal contact pressure that allows the most accurate estimation of a cardiovascular system health status on the basis of a feature of the body oscillation data.

The segmentator 432 may divide body oscillation data into predetermined time intervals, wherein the oscillation data is measured at the time when the contact pressure of the apparatus 400 for the user's body part reaches the predetermined contact pressure. Here, the predetermined time may be set to a time interval (e.g., five seconds) or may be set to a user's input value or a default value in consideration of the heartbeat cycle.

The feature calculator 433 may calculate a feature from the divided body oscillation data.

The health status estimator 434 may estimate a health status of the user's cardiovascular system using the calculated feature and the cardiovascular system health status estimation model 435.

The cardiovascular system health status estimation model 435 may define relationships between at least one feature and each cardiovascular system health status. The cardiovascular system health status estimation model 435 may be implemented in the form of a mathematical algorithm or a matching table, but is not limited to any particular form.

The actuator 440 may adjust the contact pressure of the apparatus 400 according to a control signal of the processor 430 using a pneumatic pump, a metal wire, or the like.

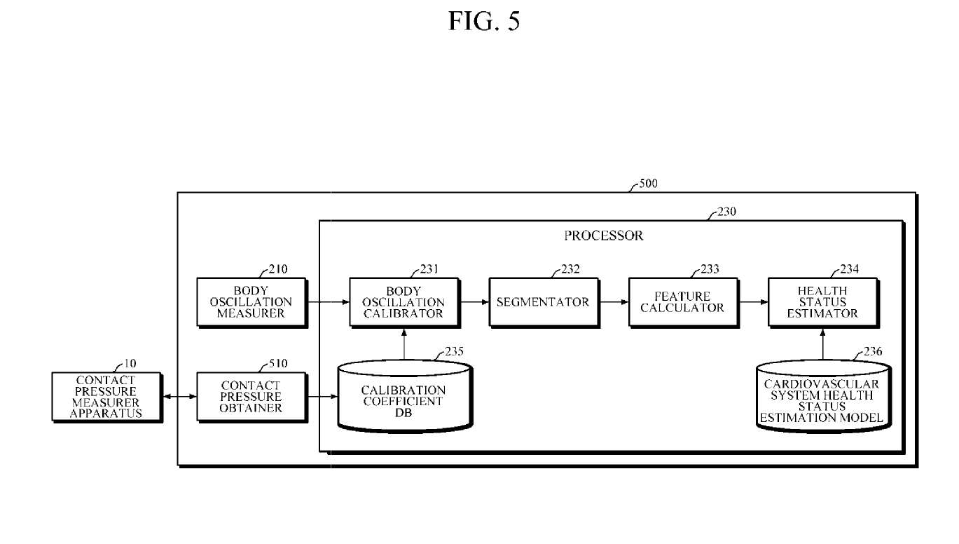

FIG. 5 is a block diagram illustrating an apparatus 500 for providing a health status of a cardiovascular system, according to still another example embodiment.

Referring to FIG. 5, the apparatus 500 for providing a health status of a cardiovascular system includes a body oscillation measurer 210, a contact pressure obtainer 510, and a processor 230. Here, the body oscillation measurer 210 and the processor 230 are the same as those described with reference to FIG. 2, and hence the detailed descriptions thereof will be omitted.

The contact pressure obtainer 510 may receive contact pressure data of the apparatus 500 in contact with a user's body part at the time of measuring body oscillation data from a contact pressure measurement apparatus 10. In this case, the contact pressure obtainer 510 may use various communication technologies, such as Bluetooth communication, Bluetooth low energy (BLE) communication, near-field communication (NFC), wireless local area network (WLAN) communication, ZigBee communication, infrared data association (IrDA) communication, Wi-Fi direct (WFD) communication, ultra-wideband (UWB) communication, Ant+ communication, Wi-Fi communication, radio frequency identification (RFID) communication, 3G communication, 4G communication, 5G communication, or the like.

The contact pressure measurement apparatus 10 is an apparatus for measuring contact pressure data of the apparatus 500 for the user's body part, and may include various cardiovascular system health status measurement apparatus (e.g., a blood pressure monitors and the like) that measure a health status of a cardiovascular system by applying a pressure to a user's wearable device or the user's body part. The contact pressure measurement apparatus 10 may include a communication interface capable of wired/wireless communication and transmit the contact pressure data measured by the contact pressure obtainer 510 via the communication interface.

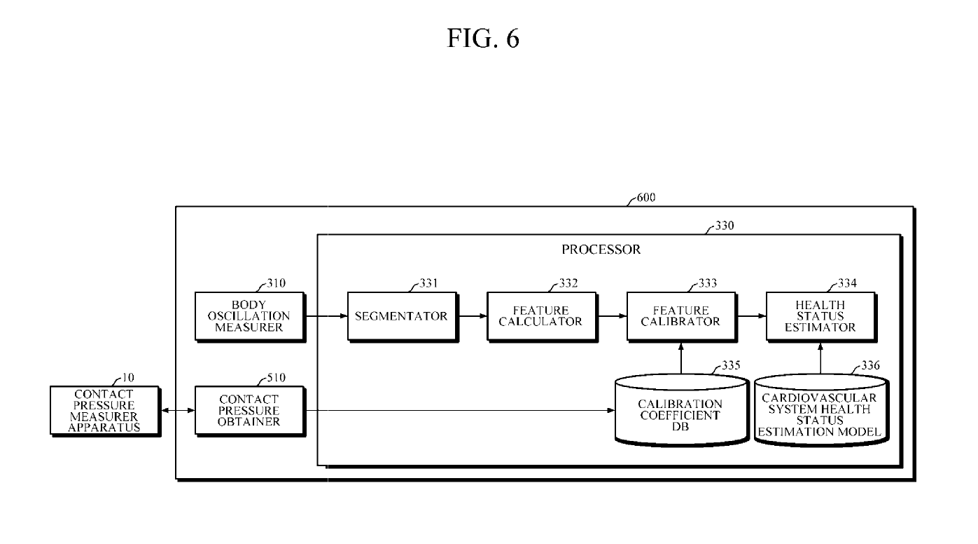

FIG. 6 is a block diagram illustrating an apparatus 600 for providing a health status of a cardiovascular system, according to yet another example embodiment.

Referring to FIG. 6, the apparatus 600 for providing a health status of a cardiovascular system includes a body oscillation measurer 310, a contact pressure obtainer 510, and a processor 330. Here, the body oscillation measurer 310 and the processor 330 are the same as those described with reference to FIG. 3, the contact pressure obtainer 510 is the same as that described with reference to FIG. 5, and thus the detailed descriptions thereof will be omitted.

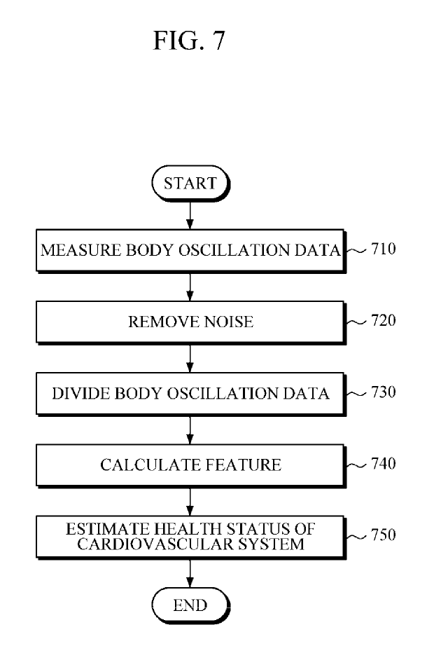

FIG. 7 is a flowchart illustrating a method of providing a health status of a cardiovascular system, according to an example embodiment.

Referring to FIGS. 1 and 7, the apparatus 100 for providing a health status of a cardiovascular system measures body oscillation data, as depicted in 710. Here, the body oscillation may refer to oscillation caused by the blood flow or pulsation of the user's body part. According to an example embodiment, the apparatus 100 may measure the body oscillation data using an acceleration sensor, a piezoelectric film, a load cell, radar, a PPG sensor, an acoustic vibration sensor, a mechanomyography sensor, or the like.

The apparatus 100 removes noise from the body oscillation data, using various noise cancellation algorithms, as depicted in 720.

The apparatus 100 divides the measured body oscillation data into predetermined time intervals, as depicted in 730. Here, the predetermined time may be set to a time interval (e.g., five seconds) or may be set to a user's input value or a default value in consideration of the heartbeat cycle.

The apparatus 100 calculates a feature from the divided body oscillation data, as depicted in 740.

The apparatus 100 may estimate a health status of the user's cardiovascular system, using the calculated feature and the cardiovascular system health status estimation model 124, as depicted in 750.

Here, the cardiovascular system health status estimation model 124 may be implemented in the form of a mathematical algorithm or a matching table, but is not limited to any particular form.

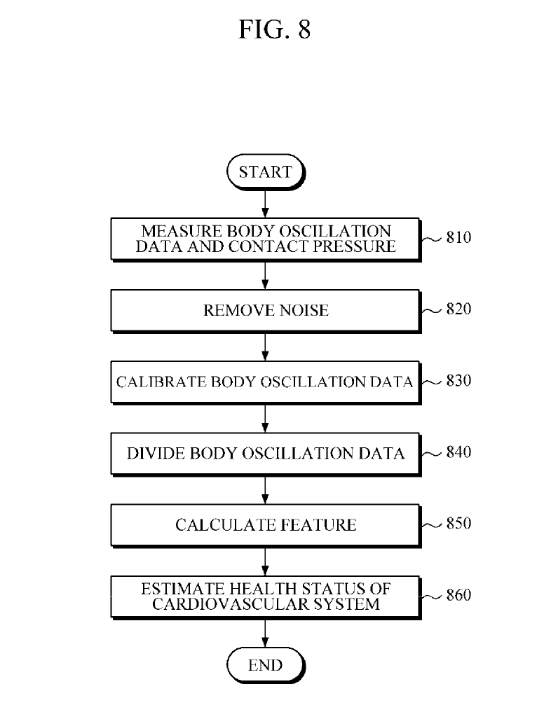

FIG. 8 is a flowchart illustrating a method of providing a health status of a cardiovascular system, according to another example embodiment.

Referring to FIGS. 2 and 8, the apparatus 200 for providing a health status of a cardiovascular system measures a user's body oscillation data and contact pressure data of the apparatus 200 in contact with the user's body part, as depicted in 810. According to an example embodiment, the apparatus 200 may measure the body oscillation data and/or the contact pressure data using the acceleration sensor, a piezoelectric film, a load cell, radar, a PPG sensor, or the like.

The apparatus 200 removes noise from the body oscillation data, using various noise cancellation algorithms, as depicted in 820.

The apparatus 200 calibrates the measured body oscillation data on the basis of the measured contact pressure data, as depicted in 830. For example, the apparatus 200 may search the calibration coefficient DB 235 to calculate a feature calibration coefficient that corresponds to the measured contact pressure data and may adjust a gain, a slope, an offset, and the like of the feature on the basis of the calculated feature calibration coefficient. The calibration coefficient DB 235 may be a set of data defining relationships between contact pressures and body oscillation calibration coefficients and may be experimentally derived and established in advance.

The apparatus 200 divides the calibrated body oscillation data into predetermined time intervals, as depicted in 840. Here, the predetermined time may be set to a time interval (e.g., five seconds) or may be set to a user's input value or a default value in consideration of the heartbeat cycle.

The apparatus 200 calculates a feature from the divided body oscillation data, as depicted in 850.

The apparatus 200 may estimate a health status of the user's cardiovascular system, using the calculated feature and the cardiovascular system health status estimation model 236, as depicted in 860.

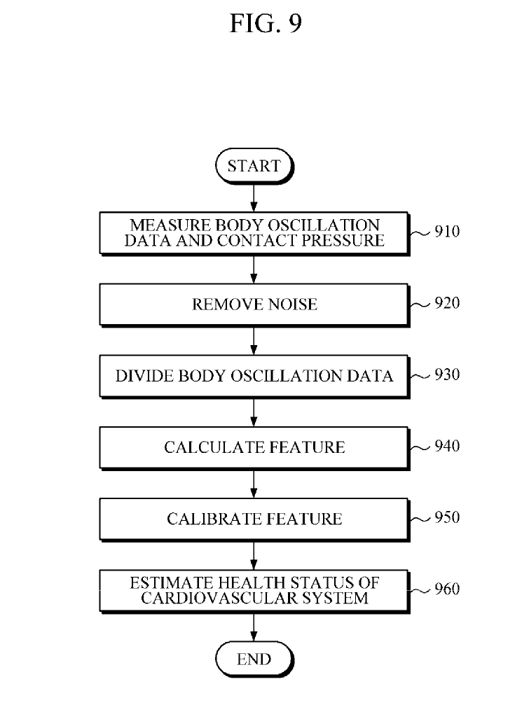

FIG. 9 is a flowchart illustrating a method of providing a health status of a cardiovascular system, according to still another example embodiment.

Referring to FIGS. 3 and 9, the apparatus 300 for providing a health status of a cardiovascular system measures a user's body oscillation data and contact pressure data of the apparatus 300 in contact with the user's body part, as depicted in 910. For example, the apparatus 300 may measure the body oscillation data and/or the contact pressure data using the acceleration sensor, a piezoelectric film, a load cell, radar, a PPG sensor, or the like.

The apparatus 300 removes noise from the body oscillation data, using various noise cancellation algorithms, as depicted in 920.

The apparatus 300 divides the measured body oscillation data into predetermined time intervals, as depicted in 930. Here, the predetermined time may be set to a time interval (e.g., 5 seconds) or may be set to a user's input value or a default value in consideration of the heartbeat cycle.

The apparatus 300 calculates a feature from the divided body oscillation data, as depicted in 940.

The apparatus 300 calibrates the calculated feature on the basis of the measured contact pressure data, as depicted in 950. For example, the apparatus 300 may search the calibration coefficient DB 335 to calculate a feature calibration coefficient that corresponds to the measured contact pressure data and may adjust a gain, a slope, an offset, and the like of the feature on the basis of the calculated feature calibration coefficient. Here, the calibration coefficient DB 335 may be a set of data defining relationships between contact pressures and body oscillation calibration coefficients and may be experimentally derived and established in advance.

The apparatus 300 estimates a health status of the user's cardiovascular system, using the calibrated feature and the cardiovascular system health state estimation model 336, as depicted in 960.

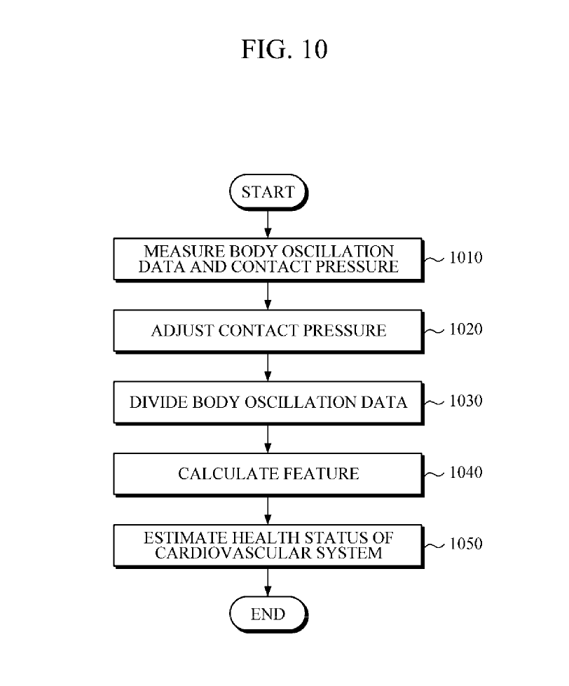

FIG. 10 is a flowchart illustrating a method of providing a health status of a cardiovascular system, according to yet another example embodiment.

Referring to FIGS. 4 and 10, the apparatus 400 for providing a health status of a cardiovascular system measures a user's body oscillation data and contact pressure data of the apparatus 400 in contact with the user's body part, as depicted in 1010. For example, the apparatus 400 may measure the body oscillation data and/or the contact pressure data using the acceleration sensor, a piezoelectric film, a load cell, radar, a PPG sensor, or the like.

The apparatus 400 adjusts the contact pressure of the apparatus 400 for the user's body part by generating a control signal for driving the actuator 440 such that the contact pressure reaches a predetermined contact pressure, as depicted in 1020. In this case, the predetermined contact pressure may be experimentally derived as an optimal contact pressure that allows the most accurate estimation of a cardiovascular system health status on the basis of a feature of the body oscillation data.

The apparatus 400 divides the measured body oscillation data into predetermined time intervals, wherein the body oscillation data is measured at the time when the contact pressure of the apparatus 400 for the user's body part reaches the predetermined contact pressure, as depicted in 1030. Here, the predetermined time may be set to a time interval (e.g., 5 seconds) or may be set to a user's input value or a default value in consideration of the heartbeat cycle.

The apparatus 400 calculates a feature from the divided body oscillation data, as depicted in 1040.

The apparatus 400 may estimate a health status of the user's cardiovascular system, using the calculated feature and the cardiovascular system health status estimation model 435, as depicted in 1050.

The cardiovascular system health status estimation model 336 may define relationships between at least one feature and each cardiovascular system health status. The cardiovascular system health status estimation model 336 may be implemented in the form of a mathematical algorithm or a matching table, but is not limited to any particular form.

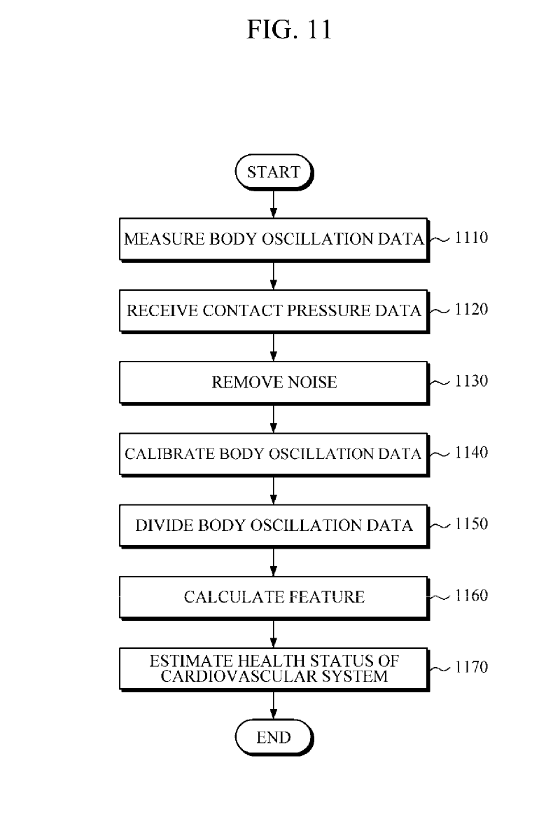

FIG. 11 is a flowchart illustrating a method of providing a health status of a cardiovascular system, according to still another example embodiment.

Referring to FIGS. 5 and 11, the apparatus 500 for providing a health status of a cardiovascular system measures a user's body oscillation data, as depicted in 1110. According to an example embodiment, the apparatus 500 may measure the body oscillation data using the acceleration sensor, a piezoelectric film, a load cell, radar, a PPG sensor, or the like.

The apparatus 500 receives contact pressure data of the apparatus 500 in contact with a user's body part at the time of measuring the body oscillation data from the contact pressure measurement apparatus 10, as depicted in 1120.

The apparatus 500 removes noise from the body oscillation data, using various noise cancellation algorithms, as depicted in 1130.

The apparatus 500 calibrates the measured body oscillation data on the basis of the received contact pressure data, as depicted in 1140. For example, the apparatus 500 may search the calibration coefficient DB 235 to calculate a feature calibration coefficient that corresponds to the measured contact pressure data and may adjust a gain, a slope, an offset, and the like of the feature on the basis of the calculated feature calibration coefficient. In this case, the calibration coefficient DB 235 may be a set of data defining relationships between contact pressures and body oscillation calibration coefficients and may be experimentally derived and established in advance.

The apparatus 500 divides the calibrated body oscillation data into predetermined time intervals, as depicted in 1150. Here, the predetermined time may be set to a time interval (e.g., 5 seconds) or may be set to a user's input value or a default value in consideration of the heartbeat cycle.

The apparatus 500 calculates a feature from the divided body oscillation data, as depicted in 1160.

The apparatus 500 may estimate a health status of the user's cardiovascular system, using the calculated feature and the cardiovascular system health status estimation model 236, as depicted in 1170.

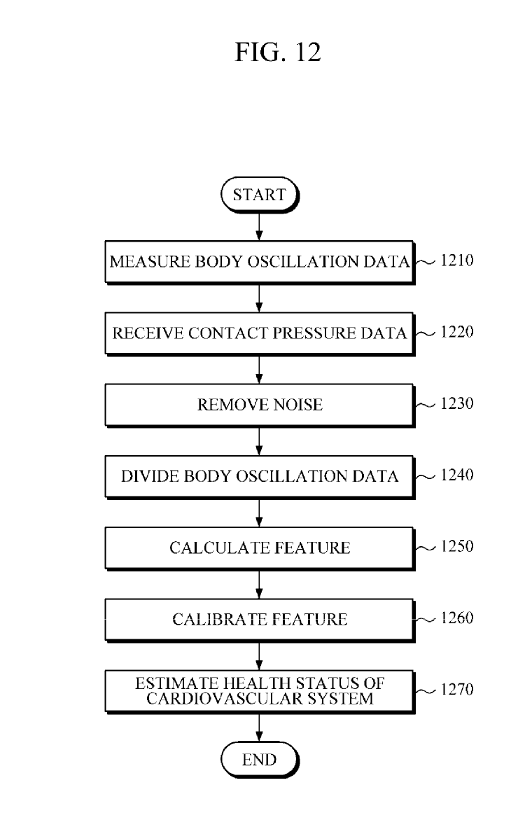

FIG. 12 is a flowchart illustrating a method of providing a health status of a cardiovascular system, according to yet another example embodiment.

Referring to FIGS. 6 and 12, the apparatus 600 for providing a health status of a cardiovascular system measures a user's body oscillation data, as depicted in 1210. According to an example embodiment, the apparatus 600 may measure the body oscillation data using the acceleration sensor, a piezoelectric film, a load cell, radar, a PPG sensor, or the like.

The apparatus 600 receives contact pressure data of the apparatus 600 in contact with a user's body part at the time of measuring the body oscillation data from the contact pressure measurement apparatus 10, as depicted in 1220.

The apparatus 600 removes noise from the body oscillation data, using various noise cancellation algorithms, as depicted in 1230.

The apparatus 600 divides the measured body oscillation data into predetermined time intervals, as depicted in 1240. Here, the predetermined time may be set to a time interval (e.g., five seconds) or may be set to a user's input value or a default value in consideration of the heartbeat cycle.

The apparatus 600 calculates a feature from the divided body oscillation data, as depicted in 1250.

The apparatus 600 calibrates the calculated feature on the basis of the received contact pressure data, as depicted in 1260. For example, the apparatus 600 may search the calibration coefficient DB 335 to calculate a feature calibration coefficient that corresponds to the measured contact pressure data and may adjust a gain, a slope, an offset, and the like of the feature on the basis of the calculated feature calibration coefficient.

The apparatus 600 estimates a health status of the user's cardiovascular system, using the calibrated feature and the cardiovascular system health state estimation model 336, as depicted in 1270.

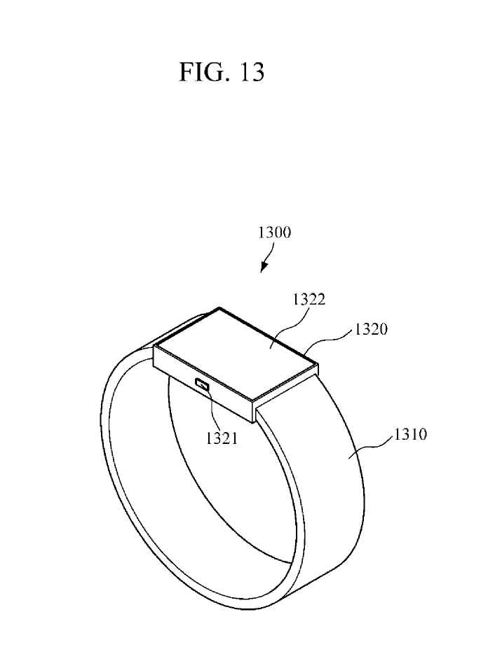

FIG. 13 is a perspective view of a wrist wearable device 1300.

Referring to FIG. 13, the wrist wearable device 1300 includes a strap 1310 and a main body 1320.

The strap 1310 may be formed in the form of a flexible band. However, aspects of the present disclosure are not limited to thereto. That is, the strap 1310 may include a plurality of strap members, each of which may be bent to wrap around the user's wrist

The main body 1320 may have the above-described apparatus 100, 200, 300, 400, 500, or 600 mounted therein. In addition, a battery for supplying power to the wrist wearable device 1300 and the apparatus 100, 200, 300, 400, 500, or 600 may be installed inside the main body 1320.

The wrist wearable device 1300 may further include an input interface 1321 and a display 1322, which are mounted in the main body 1320. The input interface 1321 may receive various operation signals from a user. The display 1322 may display data processed by the wrist wearable device 1300 and/or the apparatus 100, 200, 300, 400, 500, or 600 and processing result data.

The current example embodiments can be implemented as computer readable codes in a computer readable record medium. Codes and code segments constituting the computer program can be easily inferred by a skilled computer programmer in the art. The computer readable record medium includes all types of record media in which computer readable data are stored. Examples of the computer readable record medium include a ROM, a RAM, a CD-ROM, a magnetic tape, a floppy disk, and an optical data storage. Further, the record medium may be implemented in the form of a carrier wave such as Internet transmission. In addition, the computer readable record medium may be distributed to computer systems over a network, in which computer readable codes may be stored and executed in a distributed manner.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for providing a health status of a cardiovascular system, the apparatus comprising:
a body oscillation measurer configured to measure body oscillation data; and
a processor configured to estimate a health status of a cardiovascular system, based on the measured body oscillation data.

2. The apparatus of claim 1, wherein the body oscillation measurer is further configured to measure the body oscillation data, using any one or any combination of an acceleration sensor, a piezoelectric film, a load cell, a radar, and a photoplethysmogram sensor.

3. The apparatus of claim 1, wherein the health status of the cardiovascular system comprises any one or any combination of a blood pressure, a cardiac output, a blood vessel elasticity, and a peripheral resistance.

4. The apparatus of claim 1, wherein the processor comprises:
a segmentator configured to divide the measured body oscillation data into predetermined time intervals;
a feature calculator configured to calculate a feature from the divided body oscillation data; and
a health status estimator configured to estimate the health status of the cardiovascular system, using the calculated feature and a cardiovascular system health status estimation model.

5. The apparatus of claim 4, wherein the feature comprises any one or any combination of a signal energy, a sum of absolute values of signals, a sum of envelope values, a number of peaks, a standard deviation of amplitudes, and a number of level crossings.

6. The apparatus of claim 1, further comprising a contact pressure measurer configured to measure contact pressure data of the apparatus in contact with a body part.

7. The apparatus of claim 6, wherein the contact pressure measurer is further configured to measure the contact pressure data, using any one or any combination of an acceleration sensor, a piezoelectric film, a load cell, a radar, and a photoplethysmogram sensor.

8. The apparatus of claim 6, wherein the processor comprises:
a body oscillation calibrator configured to calibrate the measured body oscillation data, based on the measured contact pressure data;
a segmentator configured to divide the calibrated body oscillation data into predetermined time intervals;
a feature calculator configured to calculate a feature from the divided body oscillation data; and
a health status estimator configured to estimate the health status of the cardiovascular system, using the calculated feature and a cardiovascular system health status estimation model.

9. The apparatus of claim 8, wherein the body oscillation calibrator is further configured to:
search a calibration coefficient database to determine a body oscillation calibration coefficient corresponding to the measured contact pressure data; and
adjust any one or any combination of a gain, a slope, and an offset of the measured body oscillation data, based on the determined body oscillation calibration coefficient.

10. The apparatus of claim 6, wherein the processor comprises:
a segmentator configured to divide the measured body oscillation data into predetermined time intervals;
a feature calculator configured to calculate a feature from the divided body oscillation data;
a feature calibrator configured to calibrate the calculated feature, based on the measured contact pressure data; and
a health status estimator configured to estimate the health status of the cardiovascular system, using the calibrated feature and a cardiovascular system health status estimation model.

11. The apparatus of claim 10, wherein the feature calibrator is further configured to:
search a calibration coefficient database to determine a feature calibration coefficient corresponding to the measured contact pressure data; and
adjust any one or any combination of a gain, a slope, and an offset of the calculated feature, based on the determined feature calibration coefficient.

12. The apparatus of claim 6, further comprising an actuator configured to adjust a contact pressure of the apparatus in contact with the body part,
wherein the processor comprises:
a contact pressure controller configured to generate a control signal for driving the actuator such that the measured contact pressure data reaches a predetermined contact pressure,
wherein the body oscillation measurer is further configured to measure the body oscillation data in response to the measured contact pressure data reaching the predetermined contact pressure;
a segmentator configured to divide the measured body oscillation data into predetermined time intervals;
a feature calculator configured to calculate a feature from the divided body oscillation data; and
a health status estimator configured to estimate the health status of the cardiovascular system, using the calculated feature and a cardiovascular system health status estimation model.

13. The apparatus of claim 1, wherein the apparatus is implemented as a wearable device.

14. A method of providing a health status of a cardiovascular system, the method comprising:
measuring body oscillation data; and
estimating a health status of a cardiovascular system, based on the measured body oscillation data.

15. The method of claim 14, wherein the health status of the cardiovascular system comprises any one or any combination of a blood pressure, a cardiac output, a blood vessel elasticity, and a peripheral resistance.

16. The method of claim 14, further comprising:
dividing the measured body oscillation data into predetermined time intervals; and
calculating a feature from the divided body oscillation data,
wherein the estimating comprises estimating the health status of the cardiovascular system, using the calculated feature and a cardiovascular system health status estimation model.

17. The method of claim 16, wherein the feature comprises any one or any combination of a signal energy, a sum of absolute values of signals, a sum of envelope values, a number of peaks, a standard deviation of amplitudes, and a number of level crossings.

18. The method of claim 14, further comprising:
measuring contact pressure data of an apparatus in contact with a body part;
calibrating the measured body oscillation data, based on the measured contact pressure data;
dividing the calibrated body oscillation data into predetermined time intervals; and
calculating a feature from the divided body oscillation data,
wherein the estimating comprises estimating the health status of the cardiovascular system, using the calculated feature and a cardiovascular system health status estimation model.

19. The method of claim 14, further comprising:
measuring contact pressure data of an apparatus in contact with a body part;
dividing the measured body oscillation data into predetermined time intervals;
calculating a feature from the divided body oscillation data; and
calibrating the calculated feature, based on the measured contact pressure data,
wherein the estimating comprises estimating the health status of the cardiovascular system, using the calibrated feature and a cardiovascular system health status estimation model.

20. The method of claim 14, further comprising:
measuring contact pressure data of an apparatus in contact with a body part;
adjusting a contact pressure of the apparatus in contact with the body part such that the measured contact pressure data reaches a predetermined contact pressure,
wherein the measuring comprises measuring the body oscillation data in response to the measured contact pressure data reaching the predetermined contact pressure;
dividing the measured body oscillation data into predetermined time intervals; and
calculating a feature from the divided body oscillation data,
wherein the estimating comprises estimating the health status of the cardiovascular system, using the calculated feature and a cardiovascular system health status estimation model.
